(19) United States
(12) Patent Application Publication
(10) Pub. No.: US 2024/0299249 A1
(43) Pub. Date: Sep. 12, 2024

(54) Liquid Filled Capsule with Two Phase Liquid Fill

(71) Applicant: Lonza Greenwood LLC, Greenwood, SC (US)

(72) Inventors: Scarlett Singletary, Morristown, NJ (US); Stanislaw Glab, Morristown, NJ (US); Kelli Fowler, Greenwood, SC (US); Tyler White, Greenwood, SC (US)

(21) Appl. No.: 18/573,077

(22) PCT Filed: Jun. 24, 2022
(86) PCT No.: PCT/US2022/034866

§ 371 Date: Dec. 21, 2023

Related U.S. Application Data
(60) Provisional application No. 63/215,601, filed on Jun. 28, 2021.

(51) Int. Cl. A61J 1/20

(52) U.S. Cl. CPC A61J 1/2093; A61J 1/2027

(57) ABSTRACT

The present disclosure relates to an active ingredient delivery system having a container having an interior compartment and a liquid fill composition comprising A first and second liquid phase positioned in the interior compartment of the container. The first liquid phase and the second liquid phase are incompatible with each other such that the first liquid phase and the second liquid phase are immiscible and separated into two distinct phases in the interior compartment.

### BACKGROUND

Delivery of two or more active ingredients which are incompatible (immiscible) with one another in a single delivery device has been accomplished using various techniques. These techniques include, for example, placing a tablet in capsule, forming mini-tablets or beadlets of the materials and placing them in a capsule, placing one ingredient in a capsule and placing that capsule inside a larger capsule, or forming lipid (oil) multiparticulates and placing them in a capsule. Each of these methods of delivering incompatible active ingredients has advantages and disadvantages. One of the major disadvantages is cost. Costs can include the cost of making a tablets, mini-tablets, beadlets and additional capsule cost. Further, configurations such as a capsule in capsule or tablet in capsule can result in the need for larger outer capsules, which can be difficult to swallow for some users.

Other methods of placing incompatible ingredients together in a delivery form include forming an emulsion of the incompatible ingredients in a water phase and a lipid (oil) phase. These emulsions may be a water in oil emulsion or an oil in water emulsion. While emulsifying agents exist to allow 2 phase systems to be incorporated in a single capsule; the addition of additional ingredients, such as the emulsifier, cost valuable space in an already limited amount of space for ingredients. Adding the emulsifying agent may result in the need for a larger capsule to be used to deliver the active ingredients. Further, the cost of the emulsifying agent increases the cost of delivering the active ingredient to the user. Another disadvantage on using emulsifiers, is they result in fill compositions which have an opaque one phase look, which does not convey to the user that multiple ingredients are being administered to the user.

This disclosure provides a unique way to combine an aqueous phase and a lipid (oil) based phase, as well as an effective way to deliver active ingredients to a user, and in some cases a way to deliver more active ingredients to a user. The product of the present disclosure can also be visually appealing to the user. This is achieved through creating an aqueous phase that contains one or more water soluble active ingredients combined with a lipid (oil) based phase that incorporates fat-soluble actives. This provides a broad application to create a new delivery technology that is visually appealing and solves issues of immiscibility between the two phases in one dosage form. In addition to being visually appealing, it also provides for a reduced cost of manufacture and allows for a reduction of the capsule size to hold the active ingredients.

### SUMMARY

In general, the present disclosure relates to an active ingredient delivery system having a container with an interior compartment and a liquid fill composition comprising of first a liquid phase and a second liquid phase positioned in the interior compartment of the container. The first liquid phase and the second liquid phase are incompatible with each other such that the first liquid phase and the second liquid phase are immiscible and separated into two distinct phases in the interior compartment. An active ingredient present in at least one of the first liquid phase and the second liquid phase. In a specific embodiment both the first and the second liquid phases contain an active ingredient.

In one aspect of the present disclosure, the liquid fill composition of the delivery system is essentially free of an emulsifier. In addition, the liquid fill composition is not an emulsion.

In another aspect of the present disclosure, the first liquid phase is a lipid (oil)-based phase, and the second phase is an aqueous-based phase.

In an embodiment of the present disclosure, each of the first and second liquid phases contains at least one active ingredient.

In another embodiment of the present disclosure, the container is a capsule. In a typical embodiment, the capsule is a hard capsule. In a particular embodiment the container is a formed body obtained from a film forming composition comprising at least one polysaccharide or polysaccharide derivative. In specific embodiments, the polysaccharide is a cellulose derivative or pullulan.

In yet another embodiment of the present disclosure, specific embodiments of cellulose derivatives are alkyl celluloses, hydroxyalkyl celluloses, hydroxyalkylalkyl celluloses, carboxyalkyl celluloses, carboxyalkyl-alkyl celluloses, and cellulose ethers. In a particular embodiment, the cellulose derivatives are selected from the group consisting of methyl cellulose, ethyl cellulose, hydroxymethyl cellulose, hydroxyethyl cellulose, hydroxyethylethyl cellulose, hydroxypropyl cellulose, hydroxypropylmethyl cellulose (HPMC), hydroxyethylmethyl cellulose, hydroxybutylmethyl cellulose, cellulose acetylphthalate (CAP), sodium carboxymethyl cellulose, methyl cellulose ether, hydroxyethyl cellulose ether, hydroxypropyl cellulose ether, hydroxyethylmethyl cellulose ether, hydroxyethylethyl cellulose ether and hydroxypropylmethyl cellulose ether. Typically, the cellulose derivative is hydroxypropylmethyl cellulose (HPMC).

In another aspect of the present disclosure, one or both liquid phases of the liquid fill composition contain a coloring agent. In a particular aspect, the coloring agent, when present in a lipid phase is present in an amount of about 0.01 to about 10% based upon the total weight of the liquid. It is noted that in another embodiment, the active ingredient(s) may provide color to one or both phases.

In a further embodiment, the first phase is an lipid phase and the lipid (oil) phase will contain between about 0.1% by weight and about 100% by weight of the active ingredient, based on the total weight of the lipid (oil) phase, typically between about 0.5% by weight and about 75% by weight, more typically between about 1% by weight and about 20% by weight, more particularly between about 3% by weight and about 15% by weight based on the total weight of the lipid (oil) phase. In another embodiment, the second phase is a water phase, the water phase contains between 1% by weight and 75% by weight of the active ingredient, based on the total weight of the water phase, more typically between 3% by weight and 70% by weight.

In another aspect of the present disclosure provided is a method of forming a two phase liquid capsule fill composition, containing a lipid phase and an aqueous phase. The lipid phase and the aqueous phase are separate and distinct form one another. The fill composition is formed by the method comprising, 1) forming a lipid phase optionally containing a lipid soluble active ingredient, 2) forming an aqueous phase optionally containing a water soluble active ingredient, 3) mixing the lipid phase with the aqueous phase using a high shear rate mixer to form a combined liquid phase, 4) placing the combined liquid phase in a container or capsule, and 5) allowing the lipid phase and the aqueous phase separate within the container or capsule. At least one of the lipid phase and the aqueous phase contains an active ingredient.

In further aspect, the mixing is performed with a high shear mixer at a mixing speed of 100 RPM (revolutions per minute) to 15,000 RPM.

In yet another embodiment of the present disclosure, provided is a delivery system wherein the container is coated or banded.

Other features and aspects of the present disclosure are discussed in greater detail below.

### Definitions

As used herein, the terms “about,” “approximately,” or “generally,” when used to modify a value, indicates that the value can be raised or lowered by 10% and remain within the disclosed aspect.

As used herein, the phrase “essentially free” means there is none or less than 0.05% by weight of the component in the formulation. This means only trace amounts of the component may be present.

As used herein, the term “active ingredient” means an ingredient that has nutraceutical values.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, active ingredient, or a nutraceutical, to a subject as accepted as standard by the medical community. For example, the present disclosure contemplates routes of delivering or administering that include oral ingestion.

Other features and aspects of the present disclosure are discussed in greater detail below.

### DETAILED DESCRIPTION

It is to be understood by one of ordinary skill in the art that the present discussion is a description of exemplary embodiments only and is not intended as limiting the broader aspects of the present disclosure.

In the present disclosure, a liquid fill composition is placed into a container or capsule, in which a user can ingest the liquid fill composition. The selection of the container or capsule container is limited to those materials which are not soluble in water and lipid (oils). In one embodiment, the container or capsule is formed from a film forming composition comprising at least one polysaccharide or polysaccharide derivative. The polysaccharide or derivative thereof can be cellulose, cellulose derivatives, starch, modified starches, pullulan, dextran or the like and mixtures of any of the foregoing.

Suitable cellulose derivatives are selected from the group consisting of alkylcelluloses, hydroxyalkylcelluloses, hydroxyalkylalkylcelluloses, carboxyalkylcelluloses, and carboxyalkyl-alkylcelluloses including, but not limited to, members selected from the group consisting of methyl cellulose, ethyl cellulose, hydroxymethylcellulose, hydroxyethyl cellulose, hydroxyethylethyl cellulose, hydroxypropyl cellulose, hydroxypropylmethyl cellulose (HPMC), hydroxyethylmethyl cellulose, hydroxybutylmethylcellulose, cellulose acetylphtalate (CAP), sodium carboxymethyl cellulose and mixtures of any of the foregoing. Especially usable in the present disclosure is hydroxpropylmethyl cellulose (HPMC).

Additional cellulose derivatives include cellulose ethers. Suitable cellulose ethers are selected from the group consisting of alkyl- and/or hydroxyalkyl substituted cellulose ether with 1 to 4 carbon atoms in the alkyl chains and are preferably selected from the group consisting of methyl cellulose ether, hydroxyethyl cellulose ether, hydroxypropyl cellulose ether, hydroxyethylmethyl cellulose ether, hydroxyethylethyl cellulose ether, hydroxypropylmethyl cellulose ether or the like and mixtures of any of the foregoing. Especially preferred is hydroxpropylmethyl cellulose ether.

Beside starch, modified starches can be used, such as starch ethers and oxidized starches, carboxymethyl starches, hydroxyalkylated starches, and succinated starches, more particularly hydroxypropylated starch (HPS) or hydroxyethylated starch (HES) or mixtures thereof can be used as a film forming material in the film forming composition for obtaining the container according to the present invention. Modified starches disclosed in U.S. Pat. No. 6,635,275 B1 are suitable for the present invention. A desired modified starch is HPS.

In one particular embodiment the container is a formed body obtained from a film forming composition comprising at least one polysaccharide or polysaccharide derivative or mixture thereof from about 90% to 99% by weight of the final container or capsule. One particular polysaccharide useable in the present disclosure is pullulan. Pullulan is a polysaccharide polymer consisting of maltotriose units, also known as α-1,4-; α-1,6-glucan′. Three glucose units in maltotriose are connected by an α-1,4 glycosidic bond, whereas consecutive maltotriose units are connected to each other by an α-1,6 glycosidic bond. Pullulan containers or capsules useable in the present disclosure include those described in U.S. Pat. No. 10,130,587 which have a low mono-, di- and oligosaccharide content.

In an exemplary embodiment according to the present invention, the container is a hard HPMC capsule. The capsule may be a two-piece hard capsule, which can be produced by dipping method or produced by injection-molding method. The capsule can be a one compartment dosage form as well as a multiple compartment dosage form. Suitable methods for making such two-piece capsules include those methods found in the art. For example, a two-piece capsule may be made by the dipping method.

The containers or capsules according to the instant disclosure are commercially available hard capsules intended for oral administration to a human or animal being. Such hard capsules are generally manufactured from polymers by using a dip molding process and equipment. In this process, pin molds are dipped into an aqueous-based film forming composition. By subsequently gelling the composition adhered on the pins a film is formed. The film is then dried, stripped off the pins and cut to a desired length. Thus, capsule caps and bodies are obtained. Such two parts are then co-axially, telescopically joined so as to form a capsule shell. Normally, caps and bodies have a side wall, an open end, and a closed end. The length of the side wall of each of said parts is generally greater than the capsule diameter. The capsule caps and bodies are telescopically joined together so as to make their side walls partially overlap and obtain a hard capsule shell. “Partially overlap” also encompasses an embodiment wherein the side walls of caps and bodies have substantially the same length so that, when a cap and a body are telescopically joined, the side wall of said cap encases the entire side wall of said body. Unless otherwise indicated, “capsule” refers to filled capsule shells whereas “shell” specifically refers to an empty capsule.

The film forming composition for obtaining the container of the present invention may optionally comprise a suitable gelling agent selected from known gelling agents with the proviso that such gelling agent improves the gelling capability of the capsule and does not interact with the liquid fill comprising the matrix composition and/or the active agent(s). Examples of suitable gelling agents include alginic acid, sodium alginate, potassium alginate, calcium alginate, agar, carrageenan, carob gum, and gellan gum. Most desired gelling agent is gellan gum.

The capsule may optionally be coated with a suitable coating agent such as a member selected from the group consisting of cellulose acetate phthalate, polyvinyl acetate phthalate, methacrylic acid gelatines, hypromellose phthalate, hydroxypropylmethyl cellulose phthalate, hydroxyalkyl methyl cellulose phthalates, hydroxypropyl methylcellulose acetate succinate and mixtures thereof to provide e.g., enteric properties.

In a further embodiment of the invention, the capsules can additionally be sealed using means known in the art such as banding or liquid sealing technology. Examples of suitable sealing techniques are those described in WO 01/08631. In one embodiment, the capsules are sealed using a hydro alcoholic solution for example, as described in WO 01/08631, which is hereby incorporated by reference.

Commercially available capsules such as DRCAPS® and VCAPS® Plus, LICAPS® available from Lonza Consumer Health Inc, having an office in Greenwood, South Carolina, are exemplary capsules usable to deliver the fill composition

The containers or capsules have an internal compartment which is capable of holding a fill formulation containing an active ingredient. In one embodiment, the container or capsules are typically clear or are nearly clear so that the liquid contents are visible to the user. The container or capsules may have a color but will generally be completely clear.

The liquid fill composition in the container or capsules has two phases; a water based phase and a lipid (oil) base phase. These two phases are separate and distinct from each other and are incompatible (insoluble) with each other. The two phases and the fill formulation should be essentially free of an emulsifier or ingredients which may have emulsifying properties. The presence of an emulsifier, or ingredients which have emulsifying properties, will not result in a liquid fill composition with two distinct phases. This is because the emulsifier, or ingredient with emulsifying properties, will tend to reduce the surface tension between the water phase and the lipid (oil) phase. Resulting in an emulsion of the water in oil or an oil in water type, depending on the ingredients in the water phase and the lipid (oil) phase.

The water, or aqueous phase, of the fill composition contains water and at least one water soluble active ingredient. Other ingredients may be present in the water phase, provided that any additional ingredient does not have emulsifying properties. Examples of additional ingredients that may be present in the water or aqueous phase includes glycerin, gum thickening systems, or any other system that can be used to thicken the aqueous phase. Additionally, choline chloride can be used as a thinning agent for the aqueous phase.

Lipid (oil) base may be an oil based composition. The oil itself may be an active agent or the oil may contain lipid (oil) soluble active ingredient. Oils usable in the present disclosure may be a plant oil, animal oil, marine oil, or mineral oil, and/or any other edible oil. Generally, the oil should be acceptable in pharmaceuticals and/or foods.

In one embodiment, plant oil is used. Examples of plant oils include safflower oil, olive oil, soybean oil, linseed oil, rice germ oil, wheat germ oil, coconut oil, corn oil, cottonseed oil, palm oil, palm nucleus oil, peanut oil, rapeseed oil, sesame oil, sunflower oil, almond oil, cashew oil, hazelnut oil, macadamia nut oil, mongongo oil, pecan oil, pine nut oil, pistachio oil, walnut oil, calabash seed oil, buffalo gourd oil, pumpkin seed oil, watermelon seed oil, blackcurrant seed oil, borage seed oil, evening primrose oil, amaranth oil, apricot oil, apple seed oil, argan oil, artichoke oil, avocado oil, babassu oil, ben oil, cape chestnut oil, carob oil, cohune palm oil, coriander oil, dica oil, false flax oil, grape seed oil, hemp oil, kapok seed oil, lallemantia oil, marula oil, meadowfoam seed oil, mustard oil, okra seed oil (hibiscus oil), papaya oil, perilla oil, poppyseed oil, prune kernel oil, quinoa oil, ramtil oil, camellia oil, thistle oil, tomato oil, saw palmetto oil, and/or borage oil.

Animal oils include oils derived from milk fats which are the fat of cow's milk, including milk fat, butter fat, and butter oil.

Marine oils include fish oil, krill oil, docosahexaenoic acid (DHA), and eicosapentaenoic acid (EPA) derived from marine sources.

Other pharmaceutically or nutraceutically acceptable oils may be used as well, such as mineral oil and castor oil.

The water phase will have a water soluble active ingredients useable in the present disclosure include, but are not limited to, L-theanine, carnitine, Vitamin C (ascorbic acid), Vitamin B6, Vitamin B12, alpha GPC (L-Alpha glycerylphosphorylcholine), melatonin, and elderberry.

Lipid (oil) soluble active ingredients usable in the present disclosure include, but are not limited to, Vitamin E, algal oil, lavender oil, safflower oil, Vitamin A, Vitamin D, and Vitamin K. In an alternative embodiment, the oil itself in the lipid (oil) phase may be a beneficial ingredient and it is not necessary for the lipid (oil) phase to contain any additional active ingredients, such as additional lipid (oil) soluble active ingredients.

The active ingredient in the lipid (oil) phase may be up to 100% of the lipid (oil) phase. Generally, the lipid (oil) phase will contain between about 0.1% by weight and about 100% by weight of the active ingredient, based on the total weight of the lipid (oil) phase, typically between about 0.5% by weight and about 75% by weight, more typically between about 1% by weight and about 20% by weight, more particularly between about 3% by weight and about 15% by weight based on the total weight of the lipid (oil) phase, depending on the active ingredient. The upper limit of the active ingredient in the lipid (oil) phase may be up to 100% by weight of the lipid (oil) phrase, for example, up to 90% by weight, 80% by weight, 70% by weight, 60% by weight, 50% by weight, 40% by weight, 30% by weight or 20% by weight or any amount in between. The lower limit of the active ingredient in the lipid (oil) phase may be greater than or equal to 0.1% by weight, 0.5% by weight, 1% by weight, 3% by weight, 5% by weight, 7% by weight, 10% by weight, 15% by weight, 20% by weight, 30% by weight or more, and any amount in between these defined lower limits.

The active ingredient in the water phase may be present in an amount up to 100% by weight, based on the total weight of the water phase. Generally, the water phase will contain between 0.1% by weight and 80% by weight of the active ingredient, based on the total weight of the water phase, typically between 1% by weight and 75% by weight of the active ingredient, based on the total weight of the water phase, more typically in the range between 3% by weight and 70% by weight, depending on the active ingredient. The upper limit of the active ingredient in the water phase may be up to 80% by weight of the water phrase, for example up to 75% by weight, 70% by weight, 60% by weight, 50% by weight, 40% by weight, 30% by weight or 20% by weight or any amount in between. The lower limit of the active ingredient in the water phase may be greater than or equal to 0.1% by weight, 0.5% by weight, 1% by weight, 3% by weight, 5% by weight, 7% by weight, 10% by weight, 15% by weight, 20% by weight, 30% by weight or more, and any amount in between these defined lower limits.

Other ingredients may be present in each of the lipid (oil) phase and the water phase of the fill composition. Examples of other ingredients include, for example, an antioxidant, a colorant, a preservative, a flavor masking material, a surfactant which does not cause the phases to separate, a viscosity modifying agent, and mixtures thereof. Non-limiting examples of antioxidants include ascorbic acid and preservatives. Non-limiting examples of preservatives are BHA (butyl hydroxyanisole), BHT (butyl hydroxutoluene), and ascorbyl palmitate. Non-limiting examples of a surfactant include polysorbate. Non-limiting examples of viscosity modifying agents include silicon dioxide and HPMC. The other ingredients must maintain that the oil phase and the water phase remain separate in the container or capsule. Further, the additional ingredients, desirably, help the user of a capsule or container to identify the two phase present in the capsule or container. In addition, the liquid nature, both a lipid (oil) phase and an aqueous phase must not contain ingredients of the fill and must not provide deleterious effects on the hard capsule shell.

The fill composition can be dosed as a single phase formulation into the container or capsule. To form a single phase formulation, the lipid (oil) phase and the water phase are together subjected to a high shear rate mixing process, which will result in forming a single phase formulation. Typical shear rate can be 100 RPM (revolutions per minute) to 15,000 RPM, which will ensure that the water phase and the lipid (oil) phase are uniformly mixed and can be dosed into the container or capsule. Also, the use of heat should be avoided during the mixing since heat could cause a catalyze separation. Generally, the high shear mixing should occur at or near room temperature of 25 C.

Once placed into the container, given the incompatibility of the water phase and lipid (oil) phase, the two phases will start to separate from one another to form very distinct phases within the container or capsule. Generally, the lipid (oil) phase will be about 10% by volume to about 90% by volume of the fill composition and the water phase will be about 90% by volume to about 10% by volume of the fill composition. The actual ratio may depend on the amounts of the active in each of the lipid (oil) phase and the water phase and the desired amount of the active ingredients in the container or capsule.

The addition of a colorant or coloring agent in one or both of the lipid (oil) and water phases provides an aesthetically pleasing concept that closely resembles the nostalgic lava lamp, given the compatibility of the lipid (oil) phase and the aqueous phase. The colorant or coloring agent may be present, when present, amount of about 0.01% to about 10% based upon the total weight of the liquid. In some embodiments, depending on the active ingredient, the active ingredient my also serve to provide a color to one or both of the lipid (oil) and water phases.

Unless otherwise indicated, references to compatibility of the liquid fill with the container or the capsule, means that the liquid fill, after filling into a container or capsule shell, does not impact the physical stability of the capsule, i.e., the capsules remain intact and rigid without leaking, deformation, discoloration, and/or excessive softening or brittleness when observed over the indicated time period. Physical stability or physically stable relates to at least mechanical stability of said shell, more preferably to both mechanical and chemical stability of said shell. Mechanical stability can, for example, be evaluated based on maintenance of hardness of the shell and absence of leaks. Chemical stability can, for example, be evaluated based on maintenance of an appropriate dissolution profile of an encapsulated substance (for this latter aspect, reference can be made to e.g., USP-32 substance monographs).

In one embodiment, the liquid fill, after filling into a hard capsule shell as defined above, does not impact mechanical and chemical stability, beyond an acceptable level. In one embodiment, an acceptable level is a commercially acceptable level. In one embodiment, a commercially acceptable level is a level recognized by a skilled person in the field of hard capsules, preferably liquid filled hard capsules, as a satisfactory level to commercialize the carrier filled hard capsules with acceptable manufacturing costs.

In one embodiment, the liquid fill does not affect the physical stability of a hard capsule shell (mechanical or chemical) beyond an acceptable level if, when filling a batch of preferably at least 50 hard capsule shells with said liquid fill and subjecting the filled capsules to a mechanical robustness test (e.g. tube test) at a specified shell loss on drying (LOD), 0% of the capsules in said batch fail at a shell water content comprised between about 2% and about 6% LOD. Such “mechanical robustness test” demonstrates alterations in the mechanical properties of the capsule due to interactions between the fill and the shell and to evaluate the potential tendency for brittleness or softening when filled and stored at various relative humidity conditions and temperatures.

Since the containers or capsule shells of the instant disclosure are filled with substances in liquid form, it is intended that if desired the hard capsules may be sealed or banded according to conventional techniques, either by hand or via automatic banding or sealing equipment.

A number of solutions to decrease the leakage through the body-cap gap have been developed. For example, hard gelatin capsule banding with a gelatin banding solution is commonly used to prevent the content leakage during storage. Another method to decrease leakage is to seal the cap and the body of the capsule directly to each other by means of a “sealing fluid.” See, e.g., U.S. Pat. Nos. 3,071,513; 2,924,920; FR 2,118,883, EP 0152517; U.S. Pat. No. 4,756,902; FR 2 118883; EP 0152517; and U.S. Pat. No. 4,756,902. Methods of banding two piece hard capsules, as well as apparatuses for banding are disclosed, for example, in U.S. Pat. Nos. 8,181,425; 7,229,639; 7,094,425; 5,054,208; 4,940,499; 4,922,682; 4,761,932 and 4,734,149, all of which are incorporated by reference herein.

Nonetheless, certain embodiments of the present disclosure may be better understood according to the following examples, which are intended to be non-limiting and exemplary in nature.

### Example 1

A liquid fill formulation is prepared by forming a water phase and a lipid (oil) phase in separate formulation steps as shown below using the components shown in Table 1 for the water phase, and Table 2 for that oil phase. After the lipid (oil) phase and the water phase are prepared, 100 parts by weight of the oil phase and 100 parts by weight of the water phase are combined together using a high shear mixer at a shear rate of 3500 RPM, form a nearly uniform single phase mixture of the water and oil phases. The single phase mixture is then dosed into a clear capsule. The two phases of the water and lipid (oil) mixed together are allowed to sit for a period of time in the capsule and the two phases separate forming a water phase and lipid (oil) phases inside the capsule.

TABLE 1
Water Phase (naturally colored by B12)

| Ingredient | Parts by weight |
|---|---|
| Alpha GPC | 46.7 |
| Vitamin B12 | 0.30 |
| Vitamin B6 | 0.40 |
| Glycerin | 35.0 |
| Water | 17.6 |

TABLE 2
Lipid (oil) Phase

| Ingredient | Parts |
|---|---|
| AlphaMega530 | 85.7 |
| Castor Oil | 14.3 |

### Example 2

A liquid fill formulation is prepared by forming a water phase and a lipid (oil) phase in separate formulation steps, as shown below, using the components shown in Table 1 for the water phase, and Table 2 for that oil phase. After the lipid (oil) phase and the water phase are prepared, 100 parts by weight of the oil phase and 100 parts by weight of the water phase are combined together using a high shear mixer at a shear rate of 3500 RPM, form a nearly uniform single phase mixture of the water and oil phases. The single phase mixture is then dosed into a clear capsule and allowed to sit for a period of time, allowing the phases to separate into a water phase and a lipid (oil) phase. The resulting capsule has a water phase and a liquid phase within the capsule

TABLE 3
Aqueous phase

| Ingredient | Parts by weight |
|---|---|
| Elderberry | 6.635 |
| Vitamin C | 10.881 |
| Vitamin B6 | 0.341 |
| Vitamin B12 | 0.001 |
| Glycerin | 75.823 |
| Water | 6.319 |

TABLE 4
Lipid (oil) phase

| Ingredient | Parts by weight |
|---|---|
| Vitamin E | 5.91 |
| Vitamin A | 0.25 |
| Vitamin K2 | 0.29 |
| Vitamin D3 | 0.11 |
| Castor Oil | 87.94 |
| Mineral Oil | 5.50 |

### Example 3

A liquid fill formulation is prepared by forming a water phase and a lipid (oil) phase in separate formulation steps as shown below using the components shown in Table 5 for the water phase, and Table 6 for that oil phase. After the lipid (oil) phase and water phase are prepared, 100 parts by weight of the oil phase and 100 parts by weight of the water phase are combined together using a high shear mixer at a shear rate of 3500 RPM, form a nearly uniform single phase mixture of the water and oil phases. The single phase mixture is then dosed into a clear capsule and allowed to sit for a period of time, allowing the phases to separate into a water phase and a lipid (oil) phase. The resulting capsule has a water phase and a liquid phase within the capsule.

TABLE 5
Aqueous Phase

| Ingredient | Parts by weight |
|---|---|
| Melatonin | 1.46 |
| Theanine | 1.46 |
| FD&C Blue #1 | 0.01 |
| Glycerin | 90.14 |
| Water | 6.93 |

TABLE 6
Lipid (oil) phase

| Ingredient | Parts by weight |
|---|---|
| Lavender Oil | 3.90 |
| Mineral Oil | 18.18 |
| Castor Oil | 77.92 |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. An active ingredient delivery system comprising
a. a container having an interior compartment;
b. a liquid fill composition comprising a first liquid phase and a second liquid phase, wherein the first liquid phase and the second liquid phase are incompatible with each other such that the first liquid phase and the second liquid phase are immiscible with each other;
c. an active ingredient present in at least one of the first liquid phase and the second liquid phase,
wherein the liquid fill composition is contained within the interior compartment of the container.

2. The delivery system according to claim 1, wherein liquid fill composition is essentially free of an emulsifier.

3. The delivery system according to claim 1, wherein the first liquid phase is a lipid (oil)-based phase and the second phase is an aqueous-based phase.

4. The delivery system according to claim 3, wherein each of the first and second liquid phases contains at least one active ingredient.

5. The delivery system according to claim 1, wherein the liquid fill is not an emulsion.

6. The delivery system according to claim 1, wherein the container is a capsule.

7. The delivery system according to claim 6, wherein the capsule is a hard capsule.

8. The delivery system according to claim 6, wherein the container which is a formed body obtained from a film forming composition comprising at least one polysaccharide or polysaccharide derivative.

9. The delivery system according to claim 8, wherein the polysaccharide is a cellulose derivative which is selected from the group consisting of alkyl celluloses, hydroxyalkyl celluloses, hydroxyalkylalkyl celluloses, carboxyalkyl celluloses, carboxyalkyl-alkyl celluloses, and cellulose ethers.

10. The delivery system according to claim 9, wherein cellulose derivatives are selected from the group consisting of methyl cellulose, ethyl cellulose, hydroxymethyl cellulose, hydroxyethyl cellulose, hydroxyethylethyl cellulose, hydroxypropyl cellulose, hydroxypropylmethyl cellulose (HPMC), hydroxyethylmethyl cellulose, hydroxybutylmethyl cellulose, cellulose acetylphthalate (CAP), sodium carboxymethyl cellulose, methyl cellulose ether, hydroxyethyl cellulose ether, hydroxypropyl cellulose ether, hydroxyethylmethyl cellulose ether, hydroxyethylethyl cellulose ether and hydroxypropylmethyl cellulose ether.

11. The delivery system according to claim 10 wherein said cellulose derivative is selected from hydroxypropylmethyl cellulose (HPMC).

12. The delivery system according to claim 8, wherein the polysaccharide is pullulan.

13. The delivery system according to claim 1, wherein one or both liquid phases of the liquid fill composition contain a coloring agent.

14. The delivery system according to claim 13, wherein the coloring agent, when present in a one or both of the liquid phases, is present in an amount of about 0.01 to about 10% based upon the total weight of each liquid phase.

15. The delivery system according to claim 1, wherein the first phase is a lipid (oil) phase and the lipid (oil) phase will contain between 0.1% by weight and 100% by weight of the active ingredient, based on the total weight of the lipid (oil) phase, typically between 0.5% by weight and 75% by weight, more typically between 1% by weight and 20% by weight of the lipid (oil) phase.

16. The delivery system according to claim 1, wherein the second phase is a water phase, the water phase contains between 0.1% by weight and 80% by weight of the active ingredient, based on the total weight of the water phase, typically 1% and 75% and more typically between 3% by weight and 70% by weight.

17. The delivery system according to claim 1, wherein the active ingredient provides coloring to at least one of the phases.

18. A method of forming a two phase liquid capsule fill composition containing a lipid phase and an aqueous phase, wherein the lipid phase and the aqueous phase are separate and distinct form one another, said method comprising
a. forming a lipid phase containing an active ingredient,
b. forming an aqueous phase containing a water soluble active ingredient,
c. mixing the lipid phase with the aqueous phase using a high shear rate mixer to form a combined liquid phase,
d. placing the combined liquid phase in a container or capsule, and
e. allowing the lipid phase and the aqueous phase separate within the container or capsule.

19. The method of claim 18, wherein the mixing is performed with a high shear mixer at a mixing speed of 100 RPM (revolutions per minute) to 15,000 RPM.

20. A delivery system according claim 1, wherein the container is coated or banded.
